(19) United States
(12) Patent Application Publication
(10) Pub. No.: US 2024/0209387 A1
(43) Pub. Date: Jun. 27, 2024

(54) NOVEL EXPRESSION SYSTEM FOR PRODUCTION OF INDUSTRIAL ENZYMES

(71) Applicants: CORNELL UNIVERSITY, Ithaca, NY (US); Forte Protein Inc., Ithaca, NY (US)

(72) Inventor: Kathleen Hefferon, Trumansburg, NY (US)

(21) Appl. No.: 18/390,564

(22) Filed: Dec. 20, 2023

Related U.S. Application Data
(60) Provisional application No. 63/434,282, filed on Dec. 21, 2022.

(51) Int. Cl. C12N 15/82

(52) U.S. Cl. CPC C12N 15/8257; C12N 15/8205

(57) ABSTRACT

The present disclosure is directed to a small and easy to use vector system suitable to express a protein of interest in a plant. In addition, the disclosure is directed to plants comprising the vector system of the disclosure and methods for producing a protein of interest in a plant.

### CROSS-REFERENCE TO RELATED APPLICATIONS

This application claims priority to U.S. Provisional Application Ser. No. 63/434,282, filed Dec. 21, 2022, the entire contents of which are incorporated herein by reference.

### STATEMENT OF FEDERALLY SPONSORED RESEARCH

None.

### INCORPORATION BY REFERENCE OF SEQUENCE LISTING

The Sequence Listing in an XML file, named as 41598_10546_03_US_SubstituteSequenceListing.xml of 28 KB, created on Jan. 12, 2024, and submitted to the United States Patent and Trademark Office via Patent Center, is incorporated herein by reference.

### BACKGROUND OF THE INVENTION

Without limiting the scope of the invention, its background is described in connection with plant expression vectors.

In recent years, there has been considerable interest in the use of transgenic plants to generate pharmaceutical proteins. A variety of compounds has been successfully expressed in plants, including viral and bacterial antigens and vaccines and as well as many different forms of antibodies. Plants are attractive as protein factories because they can produce large volumes of products efficiently and sustainably and, under certain conditions, can have significant advantages in manufacturing costs. The possibility of producing therapeutic protein agents on an agricultural scale by “molecular farming” is extremely attractive. In addition to the scalability of the system, one of the major advantages of plants is that they possess an endomembrane system and secretory pathway that are similar to those in mammalian cells. Thus, proteins are generally efficiently assembled with appropriate post-translational modifications. These cost and scale advantages make plant-made pharmaceuticals (PMPs) very promising for both commercial pharmaceutical production and for manufacturing products destined for the developing world.

At this point, available plant transient expression systems use vectors that are too big to handle efficiently (around 10-15 Kb). Thus, there is a need for advanced transient expression vector systems with minimal elements that still lead to a high-level expression of proteins of interest.

### SUMMARY OF THE INVENTION

As embodied and broadly described herein, an aspect of the present disclosure relates to a vector system comprising:
a first nucleic acid segment comprising: in a forward direction: a first long intergenic region (LIR); a nucleic acid encoding a C2 Rep protein and optionally a C1 RepA protein; a first short intergenic region (SIR); in a reverse direction: a second long intergenic region (LIR); a first and a second 35S promoter; a 5′-AMV long synthetic 5′UTR (AMV (synJ)); a nucleic acid encoding a protein of interest; a terminator comprising an Extensin 3′ terminator, a heat shock protein (HSP) terminator, or both; and
a second nucleic acid segment comprising: in a forward direction: a third long intergenic region (LIR); a nucleic acid encoding a C2 Rep protein and optionally a C1 RepA protein; a second short intergenic region (SIR); in a reverse direction: a fourth long intergenic region (LIR); a nucleic acid encoding a nuclear shuttle protein (NSP), a nucleic acid encoding a movement protein, or both;
wherein the first, second, third and fourth LIR comprise a same nucleotide sequence of about 145 to 175 nucleotides of a geminivirus genome, wherein the first and second SIR comprise a same nucleotide sequence of the geminivirus genome, and wherein the nucleic acid encoding a C2 Rep protein on the first nucleic acid segment and the nucleic acid encoding a C2 Rep protein on the second nucleic acid segment comprise a same nucleotide sequence of the geminivirus genome. In one aspect, the length of the first, second, third and fourth LIR comprise a same nucleotide sequence of about 145 to 175, 150 to 170, 155 to 165, or 150, 151, 152, 153, 154, 155, 156, 157, 158, 159, 160, 161, 162, 162, 164, 165, 166, 167, 168, 190 or 170 nucleotides of a geminivirus genome.

In one aspect, the first nucleic acid segment and the second nucleic acid segment are on a single replicon. In another aspect, the first nucleic acid segment is on a first replicon and the second nucleic acid segment is on a second replicon. In another aspect, the first nucleic acid segment is on a first replicon and wherein the length of the first replicon is less than 2,300 bp. In another aspect, the first nucleic acid segment further comprises a nucleic acid of interest upstream from the AMV (synJ) sequence and wherein the length of the first replicon including the nucleic acid of interest is less than 5,000 bp. In another aspect, (i) the nucleic acid encoding a C2 Rep protein in the first nucleic acid segment also encodes a C1 RepA protein, and/or (ii) the nucleic acid encoding a C2 Rep protein in the second nucleic acid segment also encodes a C1 Rep In A protein. In another aspect, (i) the nucleic acid encoding a C2 Rep protein in the first nucleic acid segment does not comprise an intron and does not encode a C1 RepA protein, and/or (ii) the nucleic acid encoding a C2 Rep protein in the second nucleic acid segment does not comprise an intron and does not encode a C1 RepA protein. In another aspect, the vector system does not comprise a sequence that encodes a RepA protein. In another aspect, the first replicon is a pUC, or a binary vector selected from pBI, pCAMBIA, or pGPTV plasmid. In another aspect, the second nucleic acid segment is on a second replicon, and wherein the second replicon further comprises a selection marker and a length of about 1,000 bp. In another aspect, the first nucleic acid segment further comprises a Left (LB) and a Right Border (RB). In another aspect, the first nucleic acid segment further comprises one or more multiple cloning sites (MCS). In another aspect, the vector system further comprises a nucleic acid sequence encoding a protein of interest selected from Table 1. In another aspect, the vector system does not comprise any of the following restriction sites: EcoRI, EcoRV, KpnI, SacI, XbaI, XhoI, PstI, AccI, Pvull, or PvuI. In one aspect, the length of the first, second, third and fourth LIR comprise a same nucleotide sequence of about 145 to 175, 150 to 170, 155 to 165, or 150, 151, 152, 153, 154, 155, 156, 157, 158, 159, 160, 161, 162, 162, 164, 165, 166, 167, 168, 190 or 170 nucleotides of a geminivirus genome.

As embodied and broadly described herein, an aspect of the present disclosure relates to a plant cell comprising the vector system of the instant disclosure as described herein. In some embodiments, the plant cell is a Nicotiana benthamiana cell or a lettuce cell. In one aspect, the vector system comprises a first nucleic acid segment comprising: in a forward direction: a first long intergenic region (LIR); a nucleic acid encoding a C2 Rep protein and optionally a C1 RepA protein; a first short intergenic region (SIR); in a reverse direction: a second long intergenic region (LIR); a first and a second 35S promoter; a 5′-AMV long synthetic 5′UTR (AMV (synJ)); a nucleic acid encoding a protein of interest; a terminator comprising an Extensin 3′ terminator, a heat shock protein (HSP) terminator, or both; and a second nucleic acid segment comprising: in a forward direction: a third long intergenic region (LIR); a nucleic acid encoding a C2 Rep protein and optionally a C1 RepA protein; a second short intergenic region (SIR); in a reverse direction: a fourth long intergenic region (LIR); a nucleic acid encoding a nuclear shuttle protein (NSP), a nucleic acid encoding a movement protein, or both; wherein the first, second, third and fourth LIR comprise a same nucleotide sequence of about 160 nucleotides of a geminivirus genome, wherein the first and second SIR comprise a same nucleotide sequence of the geminivirus genome, and wherein the nucleic acid encoding a C2 Rep protein on the first nucleic acid segment and the nucleic acid encoding a C2 Rep protein on the second nucleic acid segment comprise a same nucleotide sequence of the geminivirus genome. In one aspect, the length of the first, second, third and fourth LIR comprise a same nucleotide sequence of about 145 to 175, 150 to 170, 155 to 165, or 150, 151, 152, 153, 154, 155, 156, 157, 158, 159, 160, 161, 162, 162, 164, 165, 166, 167, 168, 190 or 170 nucleotides of a geminivirus genome.

As embodied and broadly described herein, an aspect of the present disclosure relates to a method for producing a protein of interest in a plant cell comprising introducing the vector system of the instant disclosure as described herein into a plant cell; and growing the plant cell under conditions to express the protein of interest. In one aspect, the vector system is introduced into a plant cell by a gene gun. In another aspect, the vector system is introduced into a plant cell by chloroplast transformation.

As embodied and broadly described herein, an aspect of the present disclosure relates to a method of producing a protein of interest in a plant comprises transforming the vector system of the instant disclosure as described herein into agrobacterium as an agrobacterium plasmid; introducing the agrobacterium into a plant cell, plant tissue, or a whole plant via vacuum infiltration; and growing the plant cell, plant tissue, or whole plant under conditions to express the protein of interest.

### BRIEF DESCRIPTION OF THE DRAWINGS

For a more complete understanding of the features and advantages of the present invention, reference is now made to the detailed description of the invention along with the accompanying figures and in which:

FIG. 1. Schematic diagram of an exemplary vector system of this disclosure comprising a first nucleic acid segment 1 and a second nucleic acid segment 2. The first nucleic acid segment includes, in a forward direction: a first long intergenic region (“LIR1”), a nucleic acid encoding a C2 Rep protein and optionally a C1 RepA protein (“[C1]/C2”), a first short intergenic region (“SIR1”), a NcoI restriction enzyme site; and in a reverse direction: a second long intergenic region (“LIR2”), a SpeI restriction enzyme, two 35S promoters in tandem (“35S X2”), a 5′-AMV long synthetic 5′UTR (“AMV (synJ)”), a multi cloning site (“MCS”), a nucleic acid encoding a protein of interest (“insert”), a multi cloning site (“MCS”), and a terminator sequence comprising a heat shock protein (HSP) terminator and an Extensin 3′ terminator. The second nucleic acid segment includes, in a forward direction: a third long intergenic region (“LIR3”), a nucleic acid encoding a C2 Rep protein and optionally a C1 RepA protein (“[C1]/C2”), a second short intergenic region (SIR); and in a reverse direction: a fourth long intergenic region (“LIR4”), a nucleic acid encoding a nuclear shuttle protein (NSP), a nucleic acid encoding a movement protein, or both (“NSP/MP”).

FIG. 2. Protein expression of Human_Angiotensin Converting Enzyme-2 (ACE2) (98 kDa), Human Myoglobin (17 kDa), and Human Alph-Lactoalbumin (HLA) (14 kDa), using a vector as depicted in FIG. 1 (where the first segment includes a nucleic acid encoding a C2 Rep protein and a C1 RepA protein) in Nicotiana benthamiana at different days after agroinfiltration using extraction buffer 1: PBS, pH 7.4 with 1 mM EDTA and 2 mM sodium metabisulfite.

FIG. 3. Protein expression of bovine serum albumin with a His tag, bovine casein, and chicken albumin using a vector depicted in FIG. 1 (where the first segment includes a nucleic acid encoding a C2 Rep protein and a C1 RepA protein) in Nicotiana benthamiana at different days after agroinfiltration using extraction buffer 1: PBS, pH 7.4 with 1 mM EDTA and 2 mM sodium metabisulfite.

FIG. 4. Enzyme-linked immunosorbent assay (ELISA)-based quantitation of expression of ACE-2 and BSA using a vector depicted in FIG. 1 (where the first segment includes a nucleic acid encoding a C2 Rep protein and a C1 RepA protein).

FIG. 5. Dose-response curve for the ELISA-based quantitation of expression of two diverse proteins using a vector depicted in FIG. 1 (where the first segment includes a nucleic acid encoding a C2 Rep protein and a C1 RepA protein).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “about” refers to +10% of a given value.

As used herein, the phrase “35S promoter” refers to a very strong constitutive promoter responsible for the transcription of the whole Cauliflower mosaic virus (CaMV) genome. In some embodiments, the 35S promoter sequence is duplicated, and in some embodiment has at least 95%, 96%, 97%, 98%, 99% or more identical to SEQ ID NO: 4.

As used herein, the term “geminivirus” refers to a large and diverse family of plant DNA viruses, with circular single-stranded (ss) DNA genomes that replicate through circular double stranded DNA intermediates. In particular, the geminiviruses replicate via a rolling circle mechanism, analogous to that used by ssDNA plasmids of gram-positive microorganisms. The only exogenous protein required for replication is the viral replication initiation (Rep) protein encoded by a geminiviral replicase gene. This multifunctional protein initiates replication at a conserved stem loop structure found in the viral origin of replication by inducing a nick within a conserved nonanucleotide motif (TAATATTALC) found in the intergenic loop sequence. Transcription of the viral genome is bidirectional with transcription initially within the intergenic (IR) region. Rep also has functions involved in controlling the plant cell cycle, and possibly also in modulating the expression of host genes involved in DNA replication. The Rep protein can act in trans, that is, it need not be expressed by the viral replicon itself, but can be supplied from another extrachromosomal viral replicon, or even from a nuclear transgene. The cis requirements for viral replication are the viral intergenic region/s (IR), which contain sequences essential for initiation of rolling circle replication (the long intergenic region (LIR) of Mastreviruses, or the intergenic region of other geminiviruses) and synthesis of the complementary strand (the short IR (SIR) of Mastreviruses).

As used herein, the phrase “a long intergenic region (LIR)” refers to a region of a long intergenic region (LIR) of a geminivirus genome that contains a rep binding site capable of mediating excision and replication by a geminivirus Rep protein. In some embodiments, an LIR sequence is at least 95%, 96%, 97%, 98%, 99% or more identical to SEQ ID NO: 1. In some embodiments, the LIR sequence comprises about 150, about 160, about 170, about 180, about 190 or about 200 nucleotides. In some embodiments, the LIR sequence is of a length of about 150, about 160, about 170, about 180, about 190 or about 200 nucleotides. In some embodiments, the LIR sequence is of a length of not more than 240, 230, 220, 210, 200 or 190 nucleotides. The length of the first, second, third, and fourth LIR can include a same or a different nucleotide sequence of about 145 to 175, 150 to 170, 155 to 165, or 150, 151, 152, 153, 154, 155, 156, 157, 158, 159, 160, 161, 162, 162, 164, 165, 166, 167, 168, 190 or 170 nucleotides of a geminivirus genome.

As used herein, the phrase “Heat Shock Protein Terminator” or “HSP ter” is a sequence that terminates protein synthesis. In some embodiments, a HSP terminator sequence is at least 95%, 96%, 97%, 98%, 99% or more identical to SEQ ID NO: 6.

As used herein, the phrase “Extensin Terminator” or “Ext ter” is a sequence that terminates protein synthesis. In some embodiments, a HSP terminator sequence is at least 95%, 96%, 97%, 98%, 99% or more identical to SEQ ID NO: 7.

As used herein, the term “nucleic acid” has its general meaning in the art and refers to refers to a coding or non-coding nucleic sequence. Nucleic acids include DNA (deoxyribonucleic acid) and RNA (ribonucleic acid) nucleic acids. Examples of nucleic acid thus include but are not limited to DNA, mRNA, RNA, rRNA, tmRNA, miRNA, piRNA, snoRNA, and snRNA. Nucleic acids thus encompass coding and non-coding region of a genome (i.e., nuclear or mitochondrial).

The nucleic acid segments used in the instant disclosure, regardless of the length of the coding sequence itself, may be combined with other nucleic acid sequences, such as promoters, polyadenylation signals, additional restriction enzyme sites, multiple cloning sites, other coding segments, and the like. In some embodiments, a nucleic acid sequence may encode a polypeptide sequence with additional heterologous coding sequences, for example to allow for purification of the polypeptide, transport, secretion, post-translational modification, or for therapeutic benefits such as targeting or efficacy.

It is contemplated that the nucleic acid constructs of the instant disclosure may encode full-length polypeptide from any source or encode a truncated version of the polypeptide such that the transcript of the coding region represents the truncated version. The truncated transcript may then be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targeting or efficacy.

As used herein, the phrase “a 5′ alfalfa mosaic virus (AMV) SynJ” or “AMV (SynJ)) sequence” refers to a translational enhancer in plants. In some embodiments, an omega leader sequence is at least 95%, 96%, 97%, 98%, 99% or more identical to SEQ ID NO: 5.

As used herein, the phrase “a short intergenic region (SIR)” refers to the complementary strand (the short IR (SIR) of Mastreviruses) of a geminivirus genome. In some embodiments, an SIR sequence is at least 95%, 96%, 97%, 98%, 99% or more identical to SEQ ID NO: 3. In some embodiments, the SIR sequence comprises about 60, about 63, about 65 or about 70 nucleotides. In some embodiments, the SIR sequence is of a length of about 60, about 63, about 65 or about 70 nucleotides. In some embodiments, the SIR sequence is of a length of not more than 75, 74, 73, 72, 71, 70, 69, 68, 67, 66, or 65 nucleotides.

As used herein, the phrase “a Rep protein” or “genimivirus Rep protein” refers to a replication initiator protein (Rep) of a geminivirus which interacts with the origin of replication. Alternate splicing of the transcript from the complementary strand of a geminivirus genome (“C transcript”) yields mRNA's for Rep and RepA. Rep mRNA (C2) is generated when a short intron is spliced, while unspliced mRNA (C1) allows translation the C1 open reading frame to produce RepA. See, e.g., Chen et al. (Human Vaccines 7:3, 331-338, 2011). Thus, a Rep protein is also referred to herein as a “C2 Rep protein”, and a RepA protein is also referred to herein as a “C1 RepA protein”.

In some embodiments, a Rep protein of a geminivirus comprises a sequence that is at least 95%, 96%, 97%, 98%, 99% or 100% identical to SEQ ID NO: 2. In some embodiments, a Rep protein of a geminivirus comprises the sequence as set forth in SEQ ID NO: 2.

As used herein, the phrase “a Nuclear Shuttle Protein” or “NSP” refers to a gene that enables vectors to move from cell to cell in a plant, thus increasing expression. In some embodiments, the Nuclear Shuttle Protein comprises a sequence that is at least 95%, 96%, 97%, 98%, 99% or 100% identical to SEQ ID NO: 8.

As used herein, the phrase “a Movement Protein” or “MP” refers to a protein that enables the vector to move from cell to cell in a plant, thus increasing expression. In one example, the MP is a begomovirus MP. In some embodiments, the Movement Protein Protein comprises a sequence that is at least 95%, 96%, 97%, 98%, 99% or 100% identical to SEQ ID NO: 9.

As used herein, the term “vector” refers to a carrier nucleic acid molecule into which a heterologous nucleic acid sequence can be inserted for introduction into a cell where it can be replicated and expressed. Vectors include DNA, RNAs, plasmids, cosmids, viruses (bacteriophage, animal viruses, and plant viruses), and artificial chromosomes (e.g., YACs). One of skill in the art would be well equipped to construct a vector through standard recombinant techniques. Exemplary vectors include pUC vectors, pIN vectors, vectors encoding a stretch of histidines, and pGEX vectors.

The term “vector system” refers to a vector or a combination of vectors that includes multiple nucleic acid segments. The inventor of the present invention has developed a small vector system that can be easily manipulated and can be used to express a protein of interest in a plant cell. Once transfected into a plant cell, the vector system of this disclosure can replicate in the plant cell and can move from one plant cell to another due to its small size.

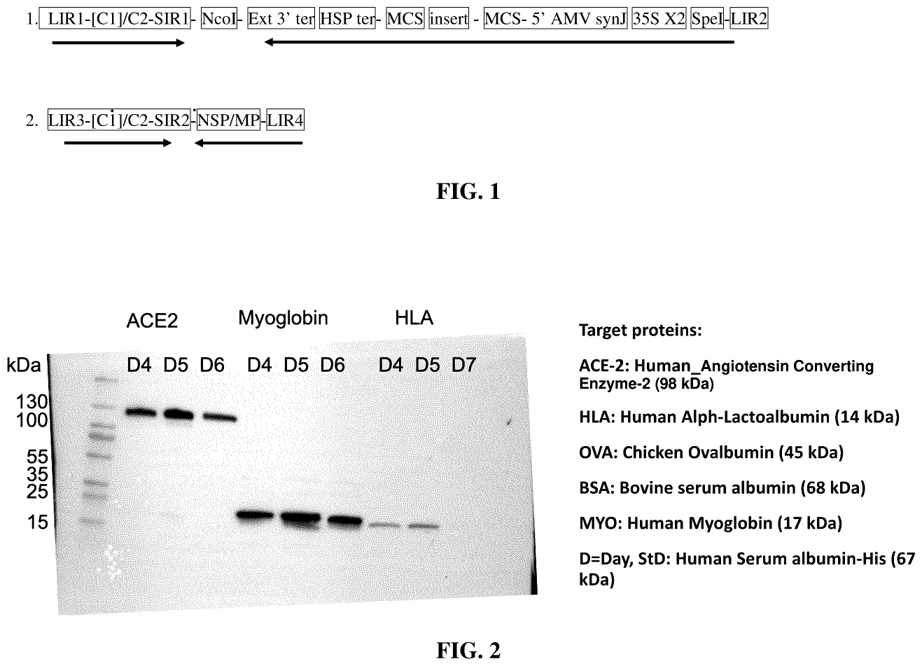

FIG. 1. Schematic diagram of an exemplary vector system of the present invention. An aspect of the instant disclosure is directed to a vector system comprising: a first and a second nucleic acid segment. In some embodiments, the vector system comprises: a first nucleic acid segment comprising: in a forward direction: a first long intergenic region (LIR); a nucleic acid encoding a C2 Rep protein and optionally a C1 RepA protein; a first short intergenic region (SIR); and in a reverse direction: a second long intergenic region (LIR); a first and a second 35S promoter; a 5′-AMV long synthetic 5′UTR (AMV (synJ)); [a multiple cloning site (MCS)]; with a nucleic acid encoding a protein of interest; [a multiple cloning site (MCS)]; and a terminator comprising an Extensin 3′ terminator, a heat shock protein (HSP) terminator, or both. A multiple cloning site (MCS) can be located on either side of the nucleic acid encoding the protein of interest, e.g., a myoglobin gene terminator. In some embodiments, the vector system comprises a second nucleic acid segment with the following elements: in a forward direction: a third long intergenic region (LIR); a nucleic acid encoding a C2 Rep protein and optionally a C1 RepA protein; a second short intergenic region (SIR); and in a reverse direction: a fourth long intergenic region (LIR); a nucleic acid encoding a nuclear shuttle protein (NSP), a nucleic acid encoding a movement protein, or both. A multiple cloning site (MCS) can be located on either side of the insert/protein of interest to be expressed. In this embodiment, the insert has already been inserted into the vector at the MCS, thus, it is shown flanked by the MCS. The vector can have one or more MCS. In some embodiments, the first, second, third and fourth LIR comprise a same nucleotide sequence of about 160 nucleotides of a geminivirus genome; the first and second SIR comprise a same nucleotide sequence of the geminivirus genome, and the nucleic acid encoding a C2 Rep protein on the first nucleic acid segment and the nucleic acid encoding a C2 Rep protein on the second nucleic acid segment comprise a same nucleotide sequence of the geminivirus genome.

In some embodiments, the first nucleic acid segment and the second nucleic acid segment are on a single replicon. In some embodiments, the first nucleic acid segment is on a first replicon and the second nucleic acid segment is on a second replicon.

In some embodiments, the first nucleic acid segment is on a first replicon and wherein the length of the first replicon, excluding any insert (i.e., nucleic acid of interest), is less than about 2500 base pairs (bp), less than 2400 bp, less than 2300 bp, or less than 2250 bp.

In some embodiments, the first nucleic acid segment further comprises a nucleic acid encoding a protein of interest after the 5′ AMV synJ and the length of the first replicon including the nucleic acid of interest is less than about 8000 bp, 7000 bp, 6000 bp, less than 5500 bp, less than 5000 bp, less than 4500 bp or less than 4000 bp, but more than 2200 bp.

In some embodiments, the nucleic acid of interest encoding a protein of interest has a length between 100 bp and 5000 bp. In some embodiments, the nucleic acid segment encoding a protein of interest is greater than 2000 bp, 2500 bp, 3000 bp, 3500 bp, 4000 bp, 4500 bp, or 5000 bp.

In some embodiments, the Rep sequence (i.e., the nucleic acid encoding a C2 Rep protein and optionally a C1 RepA protein) on either or both of the first and second nucleic acid segment does not comprise an intron. In some embodiments, the Rep sequence only encodes for a Rep protein, and does not encode for a RepA protein. In some embodiments, the vector system does not comprise a sequence that encodes a RepA protein.

In some embodiments, the first replicon is a pUC plasmid. The pBS plasmid can also be a binary vector such as pBI plasmid, pCAMBIA, or pGPTV.

In some embodiments, the second nucleic acid segment is on a second replicon, and wherein the second replicon further comprises a selection marker and a length of about 1,000 bp.

In some embodiments, the first nucleic acid segment further comprises an left border (LB) and a right border (RB) sequence.

In some embodiments, the first nucleic acid segment further comprises one or more multiple cloning sites. In some embodiments, the multiple cloning site does not comprise any of the following restriction sites: EcoRI, EcoRV, KpnI, SacI, XbaI, XhoI, PstI, AccI, PvuII, or PvuI. In some embodiments, the multiple cloning site comprises at least one restriction enzyme site selected from DraIII, SpeI, NcoI and ScaI restriction sites.

In some embodiments, the vector system further comprises a nucleic acid sequence encoding a proteint of interest, wherein the protein of interest is a protein from Table 1.

TABLE 1
Proteins that may be inserted into the vector.

| | Proteins |
|---|---|
| 1. | Human Serum Albumin |
| 2. | Bovine Serum Albumin |
| 3. | Ovalbumin |
| 4. | Collagen type I |
| 5. | Collagen type II |
| 6. | Collagen type I |
| 7. | Collagen type V |
| 8. | Thaumatin from Thaumatococcus daniellii |
| 9. | Gelatin |
| 10. | Epidermal growth factor |
| 11. | Basic fibroblast growth factor |
| 12. | Fibroblast growth factors (FGFs) |
| 13. | Acidic fibroblast growth factor |
| 14. | Leptin Chicken |
| 15. | Granulocyte-macrophage colony-stimulating |
| | factor (GMCSF) |
| 16. | Leptin Porcine |
| 17. | Transferrin, Bovine |
| 18. | Insulin, Bovine |
| 19. | Recombinant human Vascular Endothelial |
| | Growth Factor A165 (rhVEGF-A165) |
| 20. | Fibroblast growth factors -basic, Salmon |
| 21. | Gelatin Human Protein |
| 22. | Beta casein |
| 23. | Alpha-S2-Casein |
| 24. | Alpha-S1-Casein |
| 25. | Kappa (κ)- casein |
| 26. | Bovine Kappa-casein (CSN3) |
| 27. | α-lactalbumin |
| 28. | β-lactoglobulin |
| 29. | Myoglobin |
| 30. | Leghemoglobin |
| 31. | alpha-Amylase |
| 32. | Hydantoinase |
| 33. | Cyclodextrin glucanotransferase |
| 34. | Subtilase |
| 35. | Fructose bisphosphate aldolase |
| 36. | Amylosucrase |
| 37. | beta-glucosidase |
| 38. | Chymosin |
| 39. | Cyprosin |
| 40. | Lactoferrin bovine |
| 41. | Salmon collagen |
| 42. | Lactoferrin human |
| 43. | lysozyme |
| 44. | myosin |

List of fusion protein tags that may be inserted with the target protein

1. Polyhistidine (His-Tag):
Binds to metal ions (e.g., nickel) on a column.
Commonly used for immobilized metal affinity chromatography (IMAC).

2. Maltose-Binding Protein (MBP):
Binds to amylose resin, allowing for affinity chromatography.
Enhances solubility and stability of the fusion protein.

3. Glutathione S-Transferase (GST):
Binds to glutathione agarose beads.
Used in GST affinity chromatography.

4. Streptavidin/Biotin Tag:
Binds to streptavidin-coated beads or columns.
Strong interaction often used in pull-down assays.

5. Flag-Tag:
Recognized by an anti-Flag antibody.
Often used in immunoprecipitation and Western blotting.

6. HA-Tag (Human Influenza Hemagglutinin):
Recognized by an anti-HA antibody.
Commonly used in immunoprecipitation and Western blotting.

7. MYC-Tag:
Recognized by an anti-MYC antibody.
Used in immunoprecipitation and detection assays.

8. Protein A and Protein G Tags:
Recognized by antibodies.
Used for affinity purification of antibodies or antibody fragments.

9. Tandem Affinity Purification (TAP) Tag:
Consists of two tags for successive purification steps.
Improves purity in tandem affinity purification.

10. Calmodulin-Binding Peptide (CBP):
Binds to calmodulin on a resin.
Used in calmodulin affinity chromatography.

11. C-Tag:
C-tag is a 4 amino acid affinity tag: E-P-E-A (glutamic acid-proline-glutamic acid-alanine), which can be fused at the C-terminus of any recombinant protein.

12. Fc-Tag:
The Fc tag is derived from the constant region (Fc region) of an antibody. It is often used as a fusion tag to enhance the expression, solubility, and purification of a target protein.
The Fc tag can be recognized by Protein A or Protein G resins, allowing for efficient purification through affinity chromatography.

13. Small Ubiquitin-like Modifier (SUMO)
Binds to calmodulin on a resin.
Used in calmodulin affinity chromatography.

14. Thioredoxin (TRX):
Binds to calmodulin on a resin.
Used in calmodulin affinity chromatography.

15. Zein bodies:
Induce protein bodies formation when fused to a target protein
Help in encapsulation of recombinant proteins and improve yield
The proteins can be harvested with centrifugation

16. Recombinant Tobacco Etch Virus (TEV)
Binds to calmodulin on a resin. The optimum recognition site for this enzyme is the sequence Glu-Asn-Leu-Tyr-Phe-Gln-(Gly/Ser) [i.e., ENLYFQ(G/S), SEQ ID NO: 14] and cleavage occurs between the Gln and Gly/Ser residues, The most commonly used sequence is ENLYFQG (SEQ ID NO: 15).

Plant Cells. Another aspect of the disclosure is directed to a plant cell comprising the vector system of the instant disclosure as described herein. In some embodiments, the plant cell is a Nicotiana benthamiana cell or a lettuce cell.

Methods for Producing a Protein of Interest in a Plant Cell. Another aspect of the disclosure is directed to a method for producing a protein of interest in a plant cell comprising introducing the vector system of the instant disclosure as described herein into a plant cell; and growing the plant cell under conditions to express the protein of interest.

In certain aspects, the invention provides a method of producing a protein in a plant cell comprising introducing a vector system into a plant cell, wherein the vector system comprises a first nucleic acid segment comprising a promoter and a region encoding a protein of interest, wherein the region encoding a protein of interest is preceded by a sole (i.e., only one) long intergenic region (LIR) of a geminivirus genome; and a second nucleic acid segment comprising a promoter and a nucleic acid encoding a Rep protein of a geminivirus genome, wherein the second nucleic acid segment does not encode a RepA protein; and producing the protein of interest in the plant cell or a progeny of any generation thereof.

In some embodiments, introducing the vector system into the plant cell is achieved by a gene gun. In some embodiments, introducing the vector system comprises chloroplast transformation.

In some embodiments, the first nucleic acid segment and the second nucleic acid segment are comprised in respective first and second vectors. In such embodiments, the first and second vectors may be introduced into the plant cell simultaneously or separately. In other embodiments, the first nucleic acid segment and the second nucleic acid segment are comprised in a single vector.

In some embodiments, the methods of the instant disclosure further comprise isolating the protein of interest from the plant cell or a progeny of any generation thereof.

In some embodiments, a method of producing a protein of interest in a plant comprises transforming the vector system of the instant disclosure as described herein into agrobacterium as an agrobacterium plasmid; introducing the agrobacterium into a plant cell, plant tissue or whole plant via vacuum infiltration; and growing the plant cell, plant tissue, or whole plant under conditions to express the protein of interest.

Plant material: The wild-type seeds of Nicotiana benthamiana are added to a 0.8% agarose solution and then planted with help of a pipette by adding a single seed into pots containing soil in the controlled chamber or greenhouse. The seeds are grown under 16/08 hours (day/light) conditions at 250-300 μmol per meter square with maintaining 25° C.(day) and 20° C.(night) temperatures with 70% relative humidity. The plants are grown for 4-5 weeks before were agroinfiltrated.

Agrobacterium transformation: The agrobacterium EHA105 containing helper plasmid pCH32 can be used to transform the plasmids with help of an electroporator using 20052, capacitance extender 250 μFD, and capacitance 25 μFD. A total of 100 ng of plasmid DNA can be used for electroporation. The agrobacterium containing the plasmid can be grown on LB media containing antibiotics, i.e., Kanamycin and Tetracycline (10 mg/L) with 15 g of agarose for selection of the binary vectors carrying the gene of interest. The plates can be grown for two days at 28° C. in the incubator and taken out until the colonies were observed. The agrobacterium colonies can be subjected to Polymerase Chain Reaction (PCR) with primers designed for target gene sequences. Once the colonies tested positive, the individual colony can be grown in the LB media keeping the selection pressure of Kanamycin and Tetracycline (10 mg/L). The grown colonies can be centrifuged at 25° C. for 5 min and then the supernatant LB media removed and the culture resuspended into 25% glycerol solution and aliquoted for storage at 80° C.

Agrobacterium tumefaciens culture: Lysogeny broth (LB) media can be prepared with 10 g/L tryptone, 10 mg/L NaCl and 5 g/L yeast extracts into double distilled water and then autoclaved to make plates and grow Agrobacterium tumefaciens cultures containing the plasmid. The cultures can be grown initially with 5 mL volume for two days with 250 rpm rotation at 28° C. Typically, the cultures become cloudy after two days and then transferred into 250 mL LB media containing the same amount of antibiotic for selections. The overnight grown cultures can be used for agroinfiltration.

Agroinfiltration by vacuum: Before the agrobacterium cultures are processed, the 4-5 weeks grown Nicotiana benthamiana plants can be transferred from the greenhouse to the infiltration facility and kept in the dark for 1-2 hours. The agrobacterium cultures can be centrifuged at 5000×g for 5 mins to pellet agrobacterium. The supernatant is discarded, and infiltration buffer comprise of 10 mM MES buffer (pH 5.6), 10 mM MgCl2, and 150 μM of acetosyringone added to the bacterial culture. The resuspended agrobacterium cells can be diluted 100 times and then subjected to an optical density (OD) at 600 nm using a spectrophotometer. For infiltration, an OD of 0.5 is adjusted with infiltration buffer and then incubated for 1-2 hours in the dark at room temperature. Silwet L-77 can be also be added (0.05%) right before the infiltration. The Nicotiana benthamiana plants can be submerged into the infiltration buffer containing the agrobacterium and then vacuum is applied around 25 inches Hg and maintained for 3 min and then slowly released. The plants are double-checked for infiltration by visual observation and then plants are allowed to dry for 1 hour at room temperature.

Spray-agroinfiltration: The agrobacterium inoculation suspension containing 10 mM MES buffer (pH 5.6), 10 mM MgCl2, and 150 μM of acetosyringone with 0.05% Silwet-L77 can be used for spray-agroinfiltration. The bacterial solution can be applied to both sides of the Nicotiana benthamiana's leaves using a sprayer. The plants are allowed to incubate at 20° C. at 80% humidity and 16/08 hours (day/night) at 80% humidity for 9 days. The plant leaf biomass can be harvested on day 9 and collected in the zip lock bag and stored at −80° C. until used.

Post-infiltration incubation and leaf harvest: The infiltrated plants can be incubated at 20° ° C. at 80% humidity and 16/08 hours (day/night) conditions. The plants are fragile and are handled with great care to avoid any damage or leaf detachment. The plants can also be supplied with nutrient water to keep them healthy. The plant leaf can be harvested on days 5-7 to keep track of the expression of the target protein and then transferred into zip lock bags and placed on ice before the biomass is stored at −80° C.

Downstream Bioprocessing procedure: The plant material can be stored at −80° C. for at least 24 hours before they are processed for any analysis. Liquid nitrogen can be used to freeze the plant tissue for better results. The tissues can be ground with either mortar or a blender. The leaf tissues can be mixed are a 1:3 ratio with pre-chilled extraction buffer (PBS, pH 7.4 with 1 mM EDTA and 2 mM sodium metabisulfite). The ground tissues are allowed to continuous shake at 4ºC for 30 min. The crude extracts can be filtered through 6 layers of cheesecloth to remove large particles and debris. The mixture can be centrifuged at a higher speed at 30,000×g for 25 min and then filtered through 0.4 μm sterile filters. The clarified clean crude extracts can be concentrated using a Tangential flow filter (TFF) with a membrane cutoff smaller than the protein of interest. Following TFF, the concentrated crude extract can be loaded into chromatography resins for target protein enrichment. To cut down contact time and reduce the cost of production, a low-cost guard column can also be implemented in the process. The column can be connected to AKTA Pure liquid chromatography system, and concentrated plant crude extracts loaded to the column, with a flow rate ranging from 1-5 mL/min as per the manufacturer's recommendations. The washing step can be carried out using a running buffer (PBS at pH 7.4) for 8-10 column volume (CV) and then the target protein can be eluted using recommended elution buffer (PBS with 500 mM imidazole) at pH 7.4. The elution corresponding to the curve on the chromatograph can be subjected to SDS-PAGE and western blot. The highly purified elution fractions can be pooled and then dialyzed against PBS overnight at 4° C. The purified proteins can then be concentrated (10x) using a spin column with a molecular cutoff less the target protein and then stored at −80° C.

### Protein Examples.

The amino acid sequences corresponding to the target proteins can be accessed through Uniprot (https://www.uniprot.org/) and were incorporated into the vector. Codon-optimization of the DNA sequences has a positive influence on the rate of translation and mRNA degradation. Therefore, the DNA sequences can be codon-optimized for the proteins to achieve higher expression in N. benthaminana and then synthesize the plasmid, e.g., through a commercially available source such as GenScript, New Jersey, USA. The plasmids were transformed into EHA-105 Agrobacterium cells, using an electroporator following a standard protocol (Kámán-Tóth E, Pogány M, Dankó T, Szatmári A, Bozsó Z. A simplified and efficient Agrobacterium tumefaciens electroporation method. 3 Biotech. 2018 March;8(3): 148. doi: 10.1007/s13205-018-1171-9. Epub 2018 Feb 22. PMID: 29487777; PMCID: PMC5821610). The transformed cells were then grown on LB media under selection. Once the colony was confirmed for the presence of the plasmid via PCR, the colony was grown in 250 ml LB media and then 10% glycerol stocks were made and stored at −80° C.

The engineered Agrobacterium containing the plasmids were grown for 3 days and then centrifuged to harvest the cells. Before the agrobacterium cultures were processed, the 4-5 weeks of grown N. benthamiana plants were transferred from the plant growth facility to the infiltration facility and keept in the dark for 1-2 hours to help them open the stomata. The process of agroinfiltration was performed using a modified protocol as previously published (Sainsbury F, Lomonossoff GP. Extremely high-level and rapid transient protein production in plants without the use of viral replication. Plant Physiol. 2008 November; 148(3): 1212-8. doi: 10.1104/pp. 108.126284. Epub 2008 Sep 5. PMID: 18775971; PMCID: PMC2577235) using an infiltration buffer. Infiltration conditions were optimized by validating different concentrations of OD, vacuum pressure, and vacuum holding time to obtain the optimized infiltration protocol.

Spray-agroinfiltration. Generally, agroinfiltration can be carried out using various methods such as a syringe, vacuum, hydrogen peroxide, and detached leaf-based infiltration, however, vacuum infiltration is used on an industrial scale (Kaur M, Manchanda P, Kalia A, Ahmed FK, Nepovimova E, Kuca K, Abd-Elsalam KA. Agroinfiltration Mediated Scalable Transient Gene Expression in Genome Edited Crop Plants. Int J Mol Sci. 2021 Oct. 8; 22(19): 10882. doi: 10.3390/ijms221910882. PMID: 34639221; PMCID: PMC8509792). The capability of the vector was tested for penetration and cell-to-cell movement ability. In addition, it is possible to optimize infiltration conditions to improve its efficacy. For example, the bacterial solution can be applied at different concentrations to both sides of the N. benthamiana's leaves using a spray technology. The expression of recombinant proteins was estimated using Western blot and ELISA-based quantitation.

Post-infiltration incubation and leaf harvest. Post-infiltration conditions for each target protein can also be optimized by testing variables such as humidity, temperature, light intensity, light duration, and incubation time. The infiltrated plants were incubated at 18-22° C. at 50-80% humidity and different hours (day/night) conditions with various light intensities of 200-400 μmol/m2*s. The plants were supplied with nutrient water to keep them healthy. Plant leaves were harvested on days 1, 2, 3, 4, 5, 6, 7, 8 and 9 to keep track of the expression of the target protein and then stored at −80° C.

Detection and quantification of recombinant proteins. The total soluble proteins from N. benthamiana leaves were extracted with different ratios of extraction buffer i.e., 1:1, 1:3, 1:6, and 1:10. It is also possible to test different compositions of extraction buffers and investigate their impact on the recovery and functionality of our target protein. For the examples herein, the expression of the recombinant proteins was validated following a standard protocol for SDS-PAGE and Western blot as described by Spiegel et al., Current Status and Perspectives of the Molecular Farming Landscape, John Wiley & Sons, Inc., 2018. To estimate the amount of target protein in crude as well as purified samples, an ELISA assay using antibodies specified to the target proteins was used. The extraction buffer was PBS, pH 7.4 with 1 mM EDTA and 2 mM sodium metabisulfite. Following a standard protocol with modifications as published earlier by Karuppanan et al., Expression, Purification, and Biophysical Characterization of a Secreted Anthrax Decoy Fusion Protein in Nicotiana benthamiana, 2017 Int. J. Mol. Sci. 2017, 18(1), 89; doi.org/10.3390/ijms18010089. A standard Bradford assay (Bradford 1976) was used with absorbance at 280 nm to determine the total protein contents.

For accurate quantification of the target protein in the crude samples, the ELISA-based assay was used to determine the amount of recombinant proteins present. One example can be a sandwich His-tag based ELISA using antibodies against Histag. In some cases, antibodies specific to the target proteins were used to compare the quantitation.

FIG. 2 is a gel that shows the protein expression of Human_Angiotensin Converting Enzyme-2 (ACE2) (Uniprot: Q9BYF1) (98 kDa), Human Myoglobin (Uniprot: P02144) (17 kDa), and Human Alpha-Lactoalbumin (Uniprot: P00709) (HLA) (14 kDa), using a vector depicted in FIG. 1 (where the first segment includes a nucleic acid encoding a C2 Rep protein and a C1 RepA protein) in Nicotiana benthamiana at different days after agroinfiltration using extraction buffer 1: PBS, pH 7.4 with 1 mM EDTA and 2 mM sodium metabisulfite.

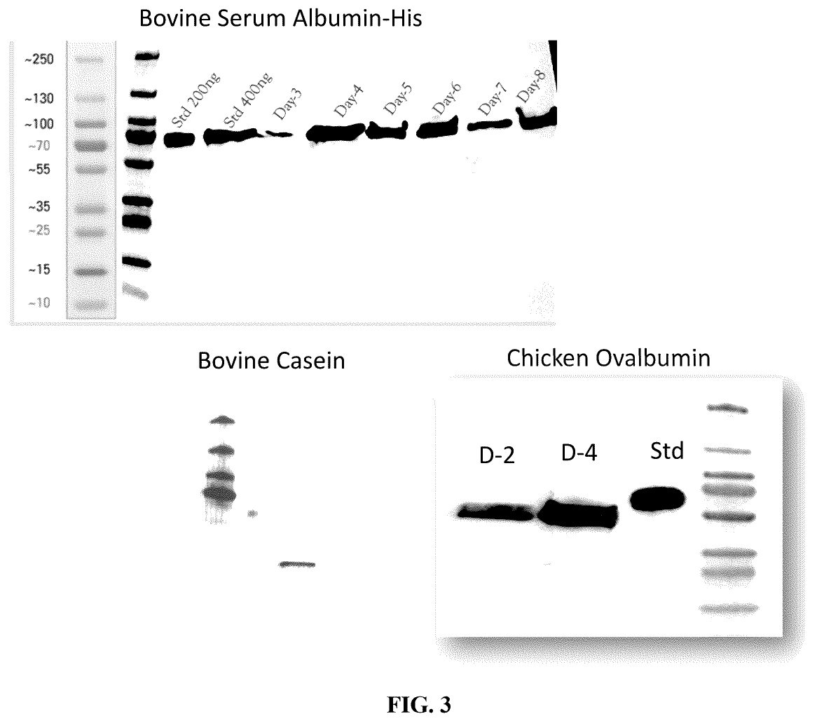

FIG. 3 shows gels with the protein expression of bovine serum albumin (Uniprot: P02769) with a His tag, bovine casein (Uniprot: P35508), and chicken albumin (Uniprot: P19121).

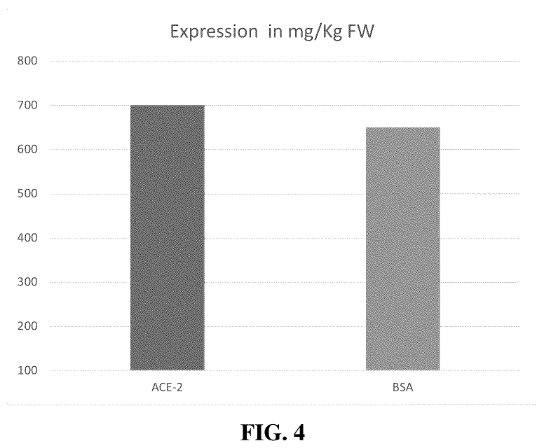

FIG. 4 is a graph that shows the results of enzyme-linked immunosorbent assay (ELISA)-based quantitation of expression of ACE-2 and BSA using the vector depicted in FIG. 1.

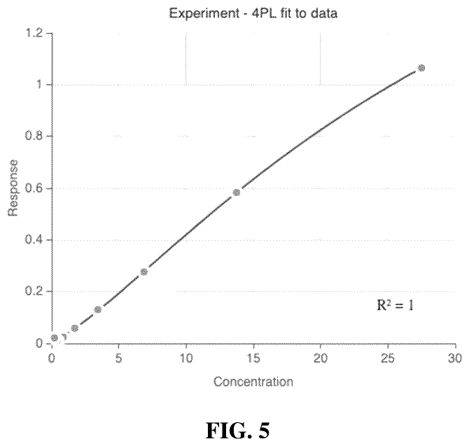

FIG. 5 is a graph with a dose-response curve for the ELISA-based quantitation of expression of two diverse proteins using a vector depicted in FIG. 1 (where the first segment includes a nucleic acid encoding a C2 Rep protein and a C1 RepA protein).

In addition to the proteins in the figures, the following proteins have also been expressed: Thaumatin (Uniprot: P02884), Chicken Ovalbumin (Uniprot: P01012), Chymosin (GenBank: J00002.1), Human Lactotransferrin (Uniprot: P02788-1) and Hemagglutinin of swine H3N2 (GenBank: V01085.1) and Alpha amylase (GenBank:AF001268.1).

Sequences. The sequences referred to in this disclosure are listed in Table 1.

| | | | SEQ |
|---|---|---|---|
| Name | Sequence | | ID NO |
| Long Internal | TGTTGTTGTGACTCCGAGGGGTTGCCTCAAACTCTATCTTATAACC | | 1 |
| Repeat (LIR) | GGCGTGGAGGCATGGAGGCAAGGGCATTTTGGTAATTTAAGTAGTT | | |
| | AGTGGAAAATGACGTCATTTACTTAAAGACGAAGTCTTGCGACAAG | | |
| | GGGGGCCCACGCCGAATTTTAATATTACCGGCGTGGCCCCACCTT | | |
| | ATCGCGAGTGCTTTAGCACGAGCGGTCCAGATTTAAAGTAGAAAAG | | |
| | TTCCCGCCCACTAGGGTTAAAGGTGTTCACACTATAAAAGCATATA | | |
| | CGATGTGATGGTATTTGA | | |
| C1/C2 | TCAGTGACTCGACGATTCCCGAGCAAAAAAAGTCTCCCCGTCACAC | | 2 |
| (complementary | ATATAGTGGGTGACGCAATTATCTTCAAAGTAATCCTTCTGTTGACT | | |
| strand) | TGTCATTGATAACATCCAGTCTTCGTCAGGATTGCAAAGAATTATAG | | |
| | AAGGGATCCCACCTTTTATTTTCTTCTTTTTTCCATATTTAGGGTTGA | | |
| | CAGTGAAATCAGACTGGCAACCTATTAATTGCTTCCACAATGGGAC | | |
| | GAACTTGAAGGGGATGTCGTCGATGATATTATAGGTGGCGTGTTCA | | |
| | TCGTAGTTGGTGAAGTCGATGGTCCCGTTCCAGTAGTTGTGTCGCC | | |
| | CGAGACTTCTAGCCCAGGTGGTCTTTCCGGTACGAGTTGGTCCGC | | |
| | AGATGTAGAGGCTGGGGTGTCTGACCCCAGTCCTTCCCTCATCCT | | |
| | GGTTAGATCGGCCATCCACTCAAGGTCAGATTGTGCTTGATCGTAG | | |
| | GAGACAGGATGTATGAAAGTGTAGGCATCGATGCTTACATGATATA | | |
| | GGTGCGTCTCTCTCCAGTTGTGCAGATCTTCGTGGCAGCGGAGAT | | |
| | CTGATTCTGTGAAGGGCGACACGTACTGCTCAGGTTGTGGAGGAA | | |
| | ATAATTTGTTGGCTGAATATTCCAGCCATTGAAGCTTTGTTGCCCAT | | |
| | TCATGAGGGAATTCTTCTTTGATCATGTCAAGATACTCCTCCTTAGA | | |
| | CGTTGCAGTCTGGATAATAGTTCGCCATCGTGCGTCAGATTTGCGA | | |
| | GGAGAGACCTTATGATCTCGGAAATCTCCTCTGGTTTTAATATCTCC | | |
| | GTCCTTTGATATGTAATCAAGGACTTGTTTAGAGTTTCTAGCTGGCT | | |
| | GGATATTAGGGTGATTTCCTTCAAAATCGAAAAAAGAAGGATCCCTA | | |
| | ATACAAGGTTTTTTATCAAGCTGGATAAGAGCATGATAGTGGGTAGT | | |
| | GCCATCTTGATGAAGCTCAGAAGCAACACCAAGGAAGAAAATAAGA | | |
| | AAAGGTGTGAGTTTCTCCCAGAGAAACTGGAATAAATCATCTCTTTG | | |
| | AGATGAGCACTTGGGGTAGGTAAGGAAAACATATTTAGATTGGAGT | | |
| | CTGAAGTTCTTGCTAGCAGAAGGCAT | | |
| Short Internal | AATGATTATTTTATGAATATATTTCATTGTGCAAGTAGATAGAAATTA | | 3 |
| Repeat (SIR) | CATATGTTACATAACACACGAAATAAACAAAAAAACACAATCCAAAA | | |
| | CAAACACCCCAAACAAAATAACACTATATATATCCTCGTATGAGGAG | | |
| | AGGCACGTTC | | |
| 35S promoter | TGAGCTTAACAGCACAGTTGCTCCTCTCAGAGCAGAATCGGGTATT | | 4 |
| X2 | CAACACCCTCATATCAACTACTACGTTGTGTATAACGGTCCACATGC | | |
| | CGGTATATACGATGACTGGGGTTGTACAAAGGCGGCAACAAACGG | | |
| | CGTTCCCGGAGTTGCACACAAGAAATTTGCCACTATTACAGAGGCA | | |
| | AGAGCAGCAGCTGACGCGTACACAACAAGTCAGCAAACAGACAGG | | |
| | TTGAACTTCATCCCCAAAGGAGAAGCTCAACTCAAGCCCAAGAGCT | | |
| | TTGCTAAGGCCCTAACAAGCCCACCAAAGCAAAAAGCCCACTGGCT | | |
| | CACGCTAGGAACCAAAAGGCCCAGCAGTGATCCAGCCCCAAAAGA | | |
| | GATCTCCTTTGCCCCGGAGATTACAATGGACGATTTCCTCTATCTTT | | |
| | ACGATCTAGGAAGGAAGTTCGAAGGTGAAGGTGACGACACTATGTT | | |
| | CACCACTGATAATGAGAAGGTTAGCCTCTTCAATTTCAGAAAGAATG | | |
| | CTGACCCACAGATGGTTAGAGAGGCCTACGCAGCAGGTCTCATCA | | |
| | AGACGATCTACCCGAGTAACAATCTCCAGGAGATCAAATACCTTCC | | |
| | CAAGAAGGTTAAAGATGCAGTCAAAAGATTCAGGACTAATTGCATC | | |
| | AAGAACACAGAGAAAGACATATTTCTCAAGATCAGAAGTACTATTCC | | |
| | AGTATGGACGATTCAAGGCTTGCTTCATAAACCAAGGCAAGTAATA | | |
| | GAGATTGGAGTCTCTAAAAAGGTAGTTCCTACTGAATCTAAGGCCA | | |
| | TGCATGGAGTCTAAGATTCAAATCGAGGATCTAACAGAACTCGCCG | | |
| | TGAAGACTGGCGAACAGTTCATACAGAGTCTTTTACGACTCAATGA | | |
| | CAAGAAGAAAATCTTCGTCAACATGGTGGAGCACGACACTCTGGTC | | |
| | TACTCCAAAAATGTCAAAGATACAGTCTCAGAAGACCAAAGGGCTA | | |
| | TTGAGACTTTTCAACAAAGGATAATTTCCGGAAACCTCCTCGGATTC | | |
| | CATTGCCCAGCTATCTGTCACTTTTATTGTGAAGATAGTGGAAAAGG | | |
| | AAGGTGGCTCCTACAAATGCCATCATTGCGATAAAGGAAAGGCCAT | | |
| | CGTTGAAGATGCCTCTGCCGACAGTGGTCCCAAAGATGGACCCCC | | |
| | ACCCACGAGGAGCATCGTGGAAAAAGAAGACGTTCCAACCACGTC | | |
| | TTCAAAGCAAGTGGATTGATGTGATATCTCCACTGACGTAAGGGAT | | |
| | GACGCACAATCCCACTATCCTTCGCAAGACCCTTCCTCTATATAAG | | |
| | GAAGTTCATTTCATTTGGAGAGGACAG | | |
| 5′ alfalfa | ACACGCTGGAATTCTAGTATACTAAACCATG | | 5 |
| mosaic virus | | | |
| (AMV) SynJ | | | |
| HSP ter | TATGAAGATGAAGATGAAATATTTGGTGTGTCAAATAAAAAGCTAGC | | 6 |
| | TTGTGTGCTTAAGTTTGTGTTTTTTTCTTGGCTTGTTGTGTTATGAAT | | |
| | TTGTGGCTTTTTCTAATATTAAATGAATGTAAGATCTCATTATAATGA | | |
| | ATAAACAAATGTTTCTATAATCCATTGTGAATGTTTTGTTGGATCTCT | | |
| | TCGCATATAACTACTGTATGTGCTATGGTATGGACTATGGAATATGA | | |
| | TTAAAGATAAG | | |
| Extensin 3′ ter | ATTGGACTCACGGCGACAGTTCAAAGGCACTAGTGGCCGACAACC | | 7 |
| | AACTAACACTCATTAATGACTTGGAAAACTGTACTGACGGGACGTTT | | |
| | TGGTCACTCCCGTCGCTTACGTCATGAGGATGTCGTCATTACAGGT | | |
| | CGAGATAGAAAATTGAAGGCTCAATTCGTTTCTTGTCATTTCACTCC | | |
| | AAAAAACGAGGAGACTATCTGTATACGGTTAAAATCGGGCCCACCC | | |
| | GATTTTACTATTTGACCGAGACTAGGAGGTTGCATCGAAGAATGGC | | |
| | CTCGTAACAGAACAGACTAAATCACGAGGATAAGGTACTGAGTTCA | | |
| | TAATCGAGGTACCGGTCGAGATCGAGGCTAGTAGTGATCGAAACC | | |
| | AAATGAGACAGACATCGAGCAAGATCGAAGATAGCACAATAACAGA | | |
| | AAGGCGAGATATCCACGACTGGTCGAGGATCATGGCATAAATCTCG | | |
| | AAACGAATCAAATTAGAAAC | | |
| Nuclear | ATGTATGGTTTGCGGAATAAACGTGGTTCATCGTTCAGCCATCGCC | | 8 |
| shuttle protein | GATTTTATTCACGTAGCAGTTTTTTAAATCGCTTGTCCGCTAATAAG | | |
| (sense strand) | CGTCATGATGGCAAACGTCGAGCTATGAATCCTAGTAAGCCCATTG | | |
| | ACGAGCCCAAGATGTCAGCCCAACGCATACATGAGAACCAGTATG | | |
| | GGCCTGAATTTGTAATGGCCCATAATTCAGCCATTTCTACGTTTATC | | |
| | AGCTACCCCAGCAAGGGCAAGATGGAACCCAACCGATCGAGGTCC | | |
| | TATATTAAGTTGAAACGACTTCGTTTCAAAGGGACTGTCAAGATTGA | | |
| | TCGTGTTCAACCAGATATGAACATTGACGGTTCTGCCCCAAAAGTG | | |
| | GAAGGAGTGTTCTCTCTGGTGGTTGTTGTGGATCGTAAACCCCACT | | |
| | TGGGTGCGTCTGGATGCCTGCATACATTCGACGAGCTGTTCGGTG | | |
| | CAAGGATCCATAGCCATGGTAATCTCAGCATAACACCCTCTTTGAA | | |
| | AGACCGATTCTACATAAGACACGTGTTCAAACGTGTATTGTCCGTG | | |
| | GAGAAGGATACGATGATGGTTGACGTGGAAGGATCTACATCGCTCT | | |
| | CTAACAGGCGATATAATTGTTGGTCCACTTTTAAGGATCTTGACCAT | | |
| | GAGTCATGCAAGGGTGTTTATGACAACATCAGCAAGAACGCCCTCC | | |
| | TAGTATATTACTGTTGGATGTCAGATACTATGTCAAAGGCATCTACT | | |
| | TTTGTATCGTTTGACCTTGATTATATCGGTTGA | | |
| Movement | TTATTGCAACGATTTGGGCTGATAAGCCTTACAGTTACTATTTATAC | | 9 |
| protein | ACTCCTGGACAGTGTTTTTCACTAGCTCGTTTAATTGCCCCATCGAC | | |
| (complementary | ATAGTAATGTTGGATTCCGCTCTCTGGGCCCCTACAATTGAGGCAG | | |
| strand) | ACTCCCCTGGGTCTAAGACGCTTGTTCCAAGCCTGCTGAGATGCCT | | |
| | ATATGGATGCATTGCGTTTTCCACCTCTGAGTCGGCATCGGAGTTG | | |
| | CTGAGCCCAATTGTACTCCGTGAAGCCCATGATTCACCCGGCTTGA | | |
| | TCTCTATTGGGCCTGGTAGTCCAATCCTTGACATGGATGCGCATCT | | |
| | TATGGGTTTCCTTTCCCATCTTCCGTAGTCGACATGTGAAAAGTCGA | | |
| | CATCTTTATCTGTGAACTGTTTCGACAGGATCTTTACTGTCGGTGCC | | |
| | CGGAAGGGTATATCCACGGAGTGTTTCGCCGTCGATAGTTTCAGTT | | |
| | TCCCTTTGAACTTGGCGAAGTGGGTCCTCTGATGAACATTCGTATC | | |
| | GCAAACCCTATAATAGAGTTTCCATGGAATTGGGTCTTTCAACGAG | | |
| | AAGAACGAAGCCGAGAAATAATGTAGATCTATGTTGCATCTGATTG | | |
| | GAAATGTCCAGGAAGCCTGCAGCGATTCGTTGTCTGTCATTCGCTT | | |
| | GTCGTGGATCTCGACAATGACCGACCCTGTCGCGTTAATCGGCACT | | |
| | TGTTGCCTGTACTCTATGACGCAGTGGTCGATCTTCATGCAGCTAC | | |
| | GGCTCAGCCTAGCTGTTAACTGCGACGCCGTGGAAGGGAATTGCA | | |
| | GAATTATCTCAGTGAGGTCATGAGAAAGTTGATATTCGTCCCGATG | | |
| | GGACTCTATGTAGTTAAATGCGTTGGGAGGATTGACCAATTGAGAA | | |
| | TCCAT | | |
| Nuclear shuttle | 1. Passionfruit leaf distortion virus isolate | | 10 |
| protein | Columbia Valle-2014-Segment B 88.64% identity | | |
| homolog 1 | www.ncbi.nlm.nih.gov/nucleotide/ | | |
| | KT899303.1?report=genbank&log$= | | |
| | nucltop&blast_rank=3&RID=SC5E7ZZJ016 | | |
| | 1 | ACCGGATGGC CGCGCATTGG TGTTCGCTCT ATCTGGTGGC CCGCGCTCTG TGATCCTGCC | |
| | 61 | ACCTGGCGCG CTCTATCGCT CCCCGTGCTC TTCTGGTGGG TCGTATTTAC TTTGACTGCC | |
| | 121 | CTTTAATTTG AATTAAAGGG CCTTTCTTTA TGTCGCGCGA GTTCTTTTGA AATTTGAATA | |
| | 181 | ATTTCCTCGC GTTATTGCCA TGGCCCACTG TACCTCACGA CTGACGTGGC TCGTTTTAGA | |
| | 241 | CCATGCGGCT GAGTCTATTT ATCTATTTTG AACCATCCTT ATCTATATAT TGAAGTCGAA | |
| | 301 | TGATATGTGT CATGTAAGCT GACTCAGCCT TCCACCACGG TTATATCGCT TTATAATTTG | |
| | 361 | TGTATTAACT GTGTTTATAT TTTGACAATG TATCCCTCGA GGAATAAACG TGGTTTGTCC | |
| | 421 | TTCACCCCAC GTCGATTTTA TGCTCGAAAC ACTGTGTTCA ACCGCCCGCA TTCTGGAAAA | |
| | 481 | CGTCAAGCCT GGAAACGTCG AGGTGCCAAT TCAAACAAGT CGGATGATGA GCCCAGAATG | |
| | 541 | TCGTCCCAAC GCATACATGA GAATCAGTAT GGGCCAGAAT TCGTTATGCC CCATAACCAA | |
| | 601 | GCCATTTCTA CGTTTATCAG CTACCCATGC GTTGGTAAGA CCGAGCCCAA CCGAAGTAGG | |
| | 661 | TCCTATATTA AGTTGAAACG ACTCCGTTTC AAGGGTACTG TGAAGATTGA ACGTGTTCAA | |
| | 721 | CCAGATATGA ACATGGATGG TTCTACCCCC AAAGTGGAAG GAGTGTTTAC TCTCGTGGTG | |
| | 781 | GTTATGGATC GCAAACCCCA CCTGGGTGCA TCTGGGTGTC TGCATACATT CGACGAGCTG | |
| | 841 | TTCGGTGCAA GGATCCATAG CCATGGTAAC CTGAGCATAA CCCCCTCTCT GAAAGACCGT | |
| | 901 | TTCTACATAC GACACGTATT CAAACGTGTA TTGTCCGTGG AGAAGGATAC GATGATGGTC | |
| | 961 | GACGTGGAAG GATCTACAAC GCTCTCTAAC AGGCGTTTTA ATTGCTGGTC CACATTCAAG | |
| | 1021 | GATCTTGATC ATGACTCATG CAACGGTGTT TATGATAACA TTAGCAAGAA CGCCCTGTTA | |
| | 1081 | GTTTATTATT GTTGGATGTC AGATACTGTG TCTAAGGCAT CAACATTTGT ATCGTTTGAC | |
| | 1141 | CTTGATTATA TAGGTTGA | |

| | | |
|---|---|---|
| Nuclear shuttle | 2. Hybarithus yellow mosaic virus DNAB 87.06% identity | 11 |
| protein | www.ncbi.nlm.nih.gov/nucleotide/NC_060098.1?report= | |
| homolog 2 | genbank&log$=nucltop&blast_rank=2&RID=SC5E7ZZJ016 | |
| | ATGTAT CCTTTGAGGG GTAAACGTGG TTTATCATTC | |
| | ACTCCACGAC GATTTTATTC ACGTAACACC GTGGTTAACC | |
| | GATCAACCGC TGGGAAACGT CATGATGGCA AACGTCGAGC | |
| | TGTGAATTCC AATAAGCCCA ATGATGAGCC CAAGATGGTA | |
| | GCCCAACGTA TACATGAGAA CCAGTATGGC CCTGAGTTTG | |
| | TCATGCCCCA TAACTCAGCC ATTTCTACGT TCATCAGCTA | |
| | TCCCAACTTG GGGAAGACCG AACCTAATCG AAGCAGGTCG | |
| | TATATCAAGT TGAAACGGCT CCGTTTCAAG GGTACTGTGA | |
| | AGATTGAACG TGTTCAATCA GATATGAACA TGGATGGTTC | |
| | TGCCCCCAAA GTGGAAGGAG TCTTCTCTCT GGTGGTGGTT | |
| | GTGGATCGTA AACCCCATTT GGGTTCATCT GGATGCTTGC | |
| | ATACATTCGA CGAGCTTTTC GGTGCAAGGA TCCATAGCCA | |
| | TGGTAATCTC AGCATAACTC CTTCCCTGAA AGACCGGTTC | |
| | TACATAAGAC ACGTGTTCAA ACGTGTGTTG TCCGTGGAGA | |
| | AGGATACGAT GATGGTCGAT GTGGAAGGAT CCACGACGCT | |
| | CTCAAACAGG CGATATAATT GTTGGTCAAC ATTCAAGGAC | |
| | CTTGACCATG ACTCATGCAA GGGTGTTTAT GACAACATTA | |
| | GCAAGAACGC CCTATTAGTG TACTACTGTT GGATGTCAGA | |
| | TACTATGTCT AAGGCATCTA CTTTTGTATC TTTCGACCTG | |
| | GATTATATTG GTTGA | |
| MP homolog 1 | Tomato leaf curl Dominican Republic virus strain segment | 12 |
| | B 84.69% | |
| | www.ncbi.nlm.nih.gov/nucleotide/NC_055515.1?report= | |
| | genbank&log$=nucltop&blast_rank=2&RID=SC6CSV43013 | |
| | AAGTT AACATTTATT TTAACGACTT GGCTTGAGAA GCCTGACAAT | |
| | TATTATTAAT ACACTCTTGG ACAGTTGTCC TGACTAGCTC | |
| | ATTCAACTGT CCCATTGACA TTGTTATGTT GGATTCCGCT | |
| | CTCTGGGCCC CCACGATCGA AGCAGACTCT CCTGGGTCTA | |
| | GGACGCTGGT CCCCAGCCTA TTCAGATGTC TGTATGGATG | |
| | GAGTTCGTTC TCCACCTCTG AGTCCGCATC TGAGTGGCCC | |
| | GTTCCTATTG TACTCCTGGA AGCCCATGAT TCTCCAGGCC | |
| | TTATCTCGAT TGGGCCTCTC AGCCCAACCC TGGACATGGA | |
| | CGCGCATCTG ATGGGCTTCC TTTCCCATTT CCCATAGTCG | |
| | ACGTGGGAGA AGTCCACATC CTTATCGGTG AACTGTTTGG | |
| | ACAGGATCTT GACTGTCGGT GCCCGGAATG GGATATCGAC | |
| | CGAGTGTTTC GCCGTCGATA GCTTCAGCTT CCCTTTGAAC | |
| | TTGGCGAAAT GGGTCCTCTG ATGAACATTC GTATCGCACA | |
| | CCCTGTAATA GAGCTTCCAT GGAATTGGGT CCTTGAGCGA | |
| | GAAGAATGAA GCCGAGAAAT AGTGGAGATC TATGTTACAT | |
| | CTGATCGGAA ATGTCCACGA CGCTTGTAAT GATTCGTTGT | |
| | CAGTCATCCT CTTGTCGTGG ATCTCCACAA TTACCGACCC | |
| | GGTGGCATTA ATCGGCACTT GCTGTCTGTA TTCTATGACG | |
| | CAATGGTCTA TCTTCATGCA GCTACGACTG AGTCTAGCTG | |
| | TCAATTGCGA CGCCGTCGAA GGAAATTGCA GTATTATCTC | |
| | AGTTAGGTCA TGGGAAAGCT GATACTCGTC CCGGTGAGAC | |
| | TCTATGTAGT TAAAGGCACT CGGAGGATTT ACTAACTGAG | |
| | AATCCAT | |
| MP homolog 2 | Tomato mosaic Trujillo virus isolate Trujillo-427b | 13 |
| | segment DNA B 84.68% identity | |
| | www.ncbi.nlm.nih.gov/nucleotide/NC_055158.1?report= | |
| | genbank&log$=nucltop&blast_rank=3&RID=SC6WXNJR016 | |
| | TAC TGCAATGATT TGGGCTGTGA AGGTCTACAA TTACTATTGA | |
| | TACACTCCTG GACAGTAGTC CTAACGAGCT CGTTCAACTG | |
| | GCCCATTGAC ATCGTGATGT TGGACTCCGC TCTCTGGGCT | |
| | CCTATAACTG ACGCAGACTC TCCTGGGTCT AGAACGGTGG | |
| | TCCCGAGCCT GCTCAGGTGT CTGTAAGGGT GGAGTTCGTT | |
| | CTCCACCTCC GAGTCCGCCT CTGAATGGCC CGTTCCTATG | |
| | GTGCTCCTGG AAGCCCACGA TTCACCAGGC CTTATCTCTA | |
| | TTGGGCCTCG AAGCCCAACC CTTGACATGG ACGCGCATCT | |
| | GATGGGCTTC CTTTCCCATC TTCCGTAGTC GACATGGGAA | |
| | AAGTCCACAT CTTTGTCCGT GAACTGTTTG GACAAGATCT | |
| | TTACTGTTGG TGCCCGGAAA GGGATGTCGA CGGAGTGTTT | |
| | CGCCGTCGAT AATTTCAGCT TCCCTTTGAA CTTCGCGAAG | |
| | TGGGTCCTCT GATGAACATT CGTGTCGCAA ACTCTGTAAT | |
| | AGAGTTTCCA TGGAATTGGG TCCTTGAGCG AGAAGAATGA | |
| | AGCTGAAAAG TAGTGGAGAT CTATGTTGCA TCTGATCGGA | |
| | AAAGTCCACG ACGCCTGCAG TGACTCGTTG TCCGTCATCC | |
| | TCTTGTCGTG GATCTCCACA ATTACCGACC CCGTCGCGTT | |
| | GATCGGAACT TGCTGCCTGT ATTCTATGAC GCAGTGGTCG | |
| | ATCTTCATGC AGCTACGACT GAGTCTAGCC GTCAACTGAG | |
| | ACGCCGTCGA CGGAAATTGC AGAACGATCT CAGTTAGGTC | |
| | ATGCGAAAGC TGATATTCGT CCCTATGGGA CTCTATGTAA | |
| | TTGAAGGCAT GCGGAGGATT AACCAACTGA GAATCCAT | |

As embodied and broadly described herein, an aspect of the present disclosure relates to a vector system comprising, consisting essentially of, or consisting of: a first nucleic acid segment comprising: in a forward direction: a first long intergenic region (LIR); a nucleic acid encoding a C2 Rep protein and optionally a C1 RepA protein; a first short intergenic region (SIR); in a reverse direction: a second long intergenic region (LIR); a first and a second 35S promoter; a 5′-AMV long synthetic 5′UTR (AMV (synJ)); a nucleic acid encoding a protein of interest; a terminator comprising an Extensin 3′ terminator, a heat shock protein (HSP) terminator, or both; and a second nucleic acid segment comprising: in a forward direction: a third long intergenic region (LIR); a nucleic acid encoding a C2 Rep protein and optionally a C1 RepA protein; a second short intergenic region (SIR); in a reverse direction: a fourth long intergenic region (LIR); a nucleic acid encoding a nuclear shuttle protein (NSP), a nucleic acid encoding a movement protein, or both; wherein the first, second, third and fourth LIR comprise a same nucleotide sequence of about 145 to 175 nucleotides of a geminivirus genome, wherein the first and second SIR comprise a same nucleotide sequence of the geminivirus genome, and wherein the nucleic acid encoding a C2 Rep protein on the first nucleic acid segment and the nucleic acid encoding a C2 Rep protein on the second nucleic acid segment comprise a same nucleotide sequence of the geminivirus genome.

In one aspect, the first nucleic acid segment and the second nucleic acid segment are on a single replicon. In another aspect, the first nucleic acid segment is on a first replicon and the second nucleic acid segment is on a second replicon. In another aspect, the first nucleic acid segment is on a first replicon and wherein the length of the first replicon is less than 2,300 bp. In another aspect, the first nucleic acid segment further comprises a nucleic acid of interest upstream from the AMV (synJ) sequence and wherein the length of the first replicon including the nucleic acid of interest is less than 5,000 bp. In another aspect, (i) the nucleic acid encoding a C2 Rep protein in the first nucleic acid segment also encodes a C1 RepA protein, and/or (ii) the nucleic acid encoding a C2 Rep protein in the second nucleic acid segment also encodes a C1 Rep In A protein. In another aspect, (i) the nucleic acid encoding a C2 Rep protein in the first nucleic acid segment does not comprise an intron and does not encode a C1 RepA protein, and/or (ii) the nucleic acid encoding a C2 Rep protein in the second nucleic acid segment does not comprise an intron and does not encode a C1 RepA protein. In another aspect, the vector system does not comprise a sequence that encodes a RepA protein. In another aspect, the first replicon is a pUC, or a binary vector selected from pBI, pCAMBIA, or pGPTV plasmid. In another aspect, the second nucleic acid segment is on a second replicon, and wherein the second replicon further comprises a selection marker and a length of about 1,000 bp. In another aspect, the first nucleic acid segment further comprises a Left (LB) and a Right Border (RB). In another aspect, the first nucleic acid segment further comprises one or more multiple cloning sites (MCS). In another aspect, the vector system further comprises a nucleic acid sequence encoding a protein of interest selected from Table 1. In another aspect, the vector system does not comprise any of the following restriction sites: FcoRI, EcoRY, KpnI, SacI, XbaI, XhoI, PstI, AccI, PvuII, or PvuI. In one aspect, the length of the first, second, third and fourth LIR comprise a same nucleotide sequence of about 145 to 175, 150 to 170, 155 to 165, or 150, 151, 152, 153, 154, 155, 156, 157, 158, 159, 160, 161, 162, 162, 164, 165, 166, 167, 168, 190 or 170 nucleotides of a geminivirus genome.

As embodied and broadly described herein, an aspect of the present disclosure relates to a plant cell comprising, consisting essentially of, or consisting of: the vector system of the instant disclosure as described herein. In some embodiments, the plant cell is a Nicotiana benthamiana cell or a lettuce cell. In one aspect, the vector system comprises a first nucleic acid segment comprising: in a forward direction: a first long intergenic region (LIR); a nucleic acid encoding a C2 Rep protein and optionally a C1 RepA protein; a first short intergenic region (SIR); in a reverse direction: a second long intergenic region (LIR); a first and a second 35S promoter; a 5′-AMV long synthetic 5′UTR (AMV (synJ)); a nucleic acid encoding a protein of interest; a terminator comprising an Extensin 3′ terminator, a heat shock protein (HSP) terminator, or both; and a second nucleic acid segment comprising: in a forward direction: a third long intergenic region (LIR); a nucleic acid encoding a C2 Rep protein and optionally a C1 RepA protein; a second short intergenic region (SIR); in a reverse direction: a fourth long intergenic region (LIR); a nucleic acid encoding a nuclear shuttle protein (NSP), a nucleic acid encoding a movement protein, or both; wherein the first, second, third and fourth LIR comprise a same nucleotide sequence of about 160 nucleotides of a geminivirus genome, wherein the first and second SIR comprise a same nucleotide sequence of the geminivirus genome, and wherein the nucleic acid encoding a C2 Rep protein on the first nucleic acid segment and the nucleic acid encoding a C2 Rep protein on the second nucleic acid segment comprise a same nucleotide sequence of the geminivirus genome.

As embodied and broadly described herein, an aspect of the present disclosure relates to a method for producing a protein of interest in a plant cell comprising, consisting essentially of, or consisting of: introducing the vector system of the instant disclosure as described herein into a plant cell; and growing the plant cell under conditions to express the protein of interest. In one aspect, the vector system is introduced into a plant cell by a gene gun. In another aspect, the vector system is introduced into a plant cell by chloroplast transformation.

As embodied and broadly described herein, an aspect of the present disclosure relates to a method of producing a protein of interest in a plant comprising, consisting essentially of, or consisting of: transforming the vector system of the instant disclosure as described herein into agrobacterium as an agrobacterium plasmid; introducing the agrobacterium into a plant cell, plant tissue, or a whole plant via vacuum infiltration; and growing the plant cell, plant tissue, or whole plant under conditions to express the protein of interest.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The various features of the methods and compositions disclosed herein are further illustrated in the manuscript attached herewith.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least #1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

For each of the claims, each dependent claim can depend from both the independent claim, and from each of the prior dependent claims for each and every claim, so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A vector system comprising:
a first nucleic acid segment comprising:
in a forward direction:
a first long intergenic region (LIR);
a nucleic acid encoding a C2 Rep protein and optionally a C1 RepA protein;
a first short intergenic region (SIR);
in a reverse direction:
a second long intergenic region (LIR);
a first and a second 35S promoter;
a 5′-AMV long synthetic 5′UTR (AMV (synJ));
a nucleic acid encoding a protein of interest;
a terminator comprising an Extensin 3′ terminator, a heat shock protein (HSP) terminator, or both; and
a second nucleic acid segment comprising:
in a forward direction:
a third long intergenic region (LIR);
a nucleic acid encoding a C2 Rep protein and optionally a C1 RepA protein;
a second short intergenic region (SIR);
in a reverse direction:
a fourth long intergenic region (LIR);
a nucleic acid encoding a nuclear shuttle protein (NSP), a nucleic acid encoding a movement protein, or both;
wherein the first, second, third and fourth LIR comprise a same nucleotide sequence of about 145 to 175 nucleotides of a geminivirus genome, wherein the first and second SIR comprise a same nucleotide sequence of the geminivirus genome, and wherein the nucleic acid encoding a C2 Rep protein on the first nucleic acid segment and the nucleic acid encoding a C2 Rep protein on the second nucleic acid segment comprise a same nucleotide sequence of the geminivirus genome.

2. The vector system of claim 1, wherein the first nucleic acid segment and the second nucleic acid segment are on a single replicon.

3. The vector system of claim 1, wherein the first nucleic acid segment is on a first replicon and the second nucleic acid segment is on a second replicon.

4. The vector system of claim 1, wherein the first nucleic acid segment is on a first replicon and wherein the length of the first replicon is less than 2,300 bp.

5. The vector system of claim 1, wherein the first nucleic acid segment further comprises a nucleic acid of interest upstream from the AMV (synJ) sequence and wherein the length of the first replicon including the nucleic acid of interest is less than 5,000 bp.

6. The vector system of claim 1, wherein (i) the nucleic acid encoding a C2 Rep protein in the first nucleic acid segment also encodes a C1 RepA protein, and/or (ii) the nucleic acid encoding a C2 Rep protein in the second nucleic acid segment also encodes a C1 RepA protein.

7. The vector system of claim 1, wherein (i) the nucleic acid encoding a C2 Rep protein in the first nucleic acid segment does not comprise an intron and does not encode a C1 RepA protein, and/or (ii) the nucleic acid encoding a C2 Rep protein in the second nucleic acid segment does not comprise an intron and does not encode a C1 RepA protein.

8. The vector system of claim 3, wherein the first replicon is a pUC, or a binary vector selected from pBI, pCAMBIA, or pGPTV plasmid.

9. The vector system of claim 3, wherein the second replicon further comprises a selection marker and a length of about 1,000 bp.

10. The vector system of claim 1, wherein the first nucleic acid segment further comprises a Left (LB) and a Right Border (RB).

11. The vector system of claim 1, wherein the first nucleic acid segment further comprises one or more multiple cloning sites (MCS).

12. The vector system of claim 1, wherein the protein of interest is selected from Table 1.

13. The vector system of claim 1, wherein the vector system does not comprise any of the following restriction sites: EcoRI, EcoRV, KpnI, SacI, XbaI, XhoI, PstI, AccI, Pull, or PvuI.

14. The vector system of claim 1, the length of the first, second, third and fourth LIR comprise the same nucleotide sequence of about 150 to 170, 155 to 165, or 150, 151, 152, 153, 154, 155, 156, 157, 158, 159, 160, 161, 162, 162, 164, 165, 166, 167, 168, 190 or 170 nucleotides of the geminivirus genome.

15. A plant cell comprising the vector system of claim 1.

16. The plant cell of claim 15, wherein the plant cell is a Nicotiana benthamiana cell.

17. The plant cell of claim 15, wherein the plant cell is a lettuce cell.

18. A method for producing a protein of interest in a plant cell comprising:
introducing the vector system of claim 1 into a plant cell; and
growing the plant cell under conditions to express the protein of interest.

19. The method of claim 18, wherein said introducing the vector system into the plant cell is achieved by a gene gun.

20. The method of claim 18, wherein said introducing the vector system comprises chloroplast transformation.

21. A method of producing a protein of interest in a plant comprising:
transforming the vector system of claim 1 into agrobacterium as an agrobacterium plasmid;
introducing the agrobacterium into a plant cell, plant tissue, or a whole plant via vacuum infiltration; and
growing the plant cell, plant tissue or the whole plant under conditions to express the protein of interest.


SEQUENCE LISTING (15 sequences)

SEQ ID NO: 1 - skipped

SEQ ID NO: 2 - DNA, 1091 bp, synthetic construct
  misc_feature 1..1091: note = Synthetic polynucleotide-C1/C2 (complementary strand)
  source 1..1091: mol_type = other DNA
tcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaattcttctttgatcatgtcaagatactcctccttagacgttgcagtctggataatagttcgccatcgtgcgtcagatttgcgaggagagaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcat

SEQ ID NO: 3 - DNA, 152 bp, synthetic construct
  misc_feature 1..152: note = Synthetic polynucleotide-Short Internal Repeat (SIR)
  source 1..152: mol_type = other DNA
aatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttc

SEQ ID NO: 4 - DNA, 1315 bp, synthetic construct
  misc_feature 1..1315: note = Synthetic polynucleotide-35S promoter X2
  source 1..1315: mol_type = other DNA
tgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttccggaaacctcctcggattccattgcccagctatctgtcacttttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacag

SEQ ID NO: 5 - DNA, 31 bp, synthetic construct
  misc_feature 1..31: note = Synthetic polynucleotide-5' alfalfa mosaic virus (AMV) SynJ
  source 1..31: mol_type = other DNA
acacgctggaattctagtatactaaaccatg

SEQ ID NO: 6 - DNA, 249 bp, synthetic construct
  misc_feature 1..249: note = Synthetic polynucleotide-HSP ter
  source 1..249: mol_type = other DNA
tatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataag

SEQ ID NO: 7 - DNA, 480 bp, synthetic construct
  misc_feature 1..480: note = Synthetic polynucleotide-Extensin 3' ter
  source 1..480: mol_type = other DNA
attggactcacggcgacagttcaaaggcactagtggccgacaaccaactaacactcattaatgacttggaaaactgtactgacgggacgttttggtcactcccgtcgcttacgtcatgaggatgtcgtcattacaggtcgagatagaaaattgaaggctcaattcgtttcttgtcatttcactccaaaaaacgaggagactatctgtatacggttaaaatcgggcccacccgattttactatttgaccgagactaggaggttgcatcgaagaatggcctcgtaacagaacagactaaatcacgaggataaggtactgagttcataatcgaggtaccggtcgagatcgaggctagtagtgatcgaaaccaaatgagacagacatcgagcaagatcgaagatagcacaataacagaaaggcgagatatccacgactggtcgaggatcatggcataaatctcgaaacgaatcaaattagaaac

SEQ ID NO: 8 - DNA, 771 bp, synthetic construct
  misc_feature 1..771: note = Synthetic polynucleotide-Nuclear shuttle protein (sense strand)
  source 1..771: mol_type = other DNA
atgtatggtttgcggaataaacgtggttcatcgttcagccatcgccgattttattcacgtagcagttttttaaatcgcttgtccgctaataagcgtcatgatggcaaacgtcgagctatgaatcctagtaagcccattgacgagcccaagatgtcagcccaacgcatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgatcgtgttcaaccagatatgaacattgacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccctctttgaaagaccgattctacataagacacgtgttcaaacgtgtattgtccgtggagaaggatacgatgatggttgacgtggaaggatctacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttga

SEQ ID NO: 9 - DNA, 882 bp, synthetic construct
  misc_feature 1..882: note = Synthetic polynucleotide-Movement protein (complementary strand)
  source 1..882: mol_type = other DNA
ttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggtttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccat

SEQ ID NO: 10 - DNA, 1158 bp, synthetic construct
  misc_feature 1..1158: note = Synthetic polynucleotide-Nuclear shuttle protein homolog 1
  source 1..1158: mol_type = other DNA
accggatggccgcgcattggtgttcgctctatctggtggcccgcgctctgtgatcctgccacctggcgcgctctatcgctccccgtgctcttctggtgggtcgtatttactttgactgccctttaatttgaattaaagggcctttctttatgtcgcgcgagttcttttgaaatttgaataatttcctcgcgttattgccatggcccactgtacctcacgactgacgtggctcgttttagaccatgcggctgagtctatttatctattttgaaccatccttatctatatattgaagtcgaatgatatgtgtcatgtaagctgactcagccttccaccacggttatatcgctttataatttgtgtattaactgtgtttatattttgacaatgtatccctcgaggaataaacgtggtttgtccttcaccccacgtcgattttatgctcgaaacactgtgttcaaccgcccgcattctggaaaacgtcaagcctggaaacgtcgaggtgccaattcaaacaagtcggatgatgagcccagaatgtcgtcccaacgcatacatgagaatcagtatgggccagaattcgttatgccccataaccaagccatttctacgtttatcagctacccatgcgttggtaagaccgagcccaaccgaagtaggtcctatattaagttgaaacgactccgtttcaagggtactgtgaagattgaacgtgttcaaccagatatgaacatggatggttctacccccaaagtggaaggagtgtttactctcgtggtggttatggatcgcaaaccccacctgggtgcatctgggtgtctgcatacattcgacgagctgttcggtgcaaggatccatagccatggtaacctgagcataaccccctctctgaaagaccgtttctacatacgacacgtattcaaacgtgtattgtccgtggagaaggatacgatgatggtcgacgtggaaggatctacaacgctctctaacaggcgttttaattgctggtccacattcaaggatcttgatcatgactcatgcaacggtgtttatgataacattagcaagaacgccctgttagtttattattgttggatgtcagatactgtgtctaaggcatcaacatttgtatcgtttgaccttgattatataggttga

SEQ ID NO: 11 - DNA, 771 bp, synthetic construct
  misc_feature 1..771: note = Synthetic polynucleotide-Nuclear shuttle protein homolog 2
  source 1..771: mol_type = other DNA
atgtatcctttgaggggtaaacgtggtttatcattcactccacgacgattttattcacgtaacaccgtggttaaccgatcaaccgctgggaaacgtcatgatggcaaacgtcgagctgtgaattccaataagcccaatgatgagcccaagatggtagcccaacgtatacatgagaaccagtatggccctgagtttgtcatgccccataactcagccatttctacgttcatcagctatcccaacttggggaagaccgaacctaatcgaagcaggtcgtatatcaagttgaaacggctccgtttcaagggtactgtgaagattgaacgtgttcaatcagatatgaacatggatggttctgcccccaaagtggaaggagtcttctctctggtggtggttgtggatcgtaaaccccatttgggttcatctggatgcttgcatacattcgacgagcttttcggtgcaaggatccatagccatggtaatctcagcataactccttccctgaaagaccggttctacataagacacgtgttcaaacgtgtgttgtccgtggagaaggatacgatgatggtcgatgtggaaggatccacgacgctctcaaacaggcgatataattgttggtcaacattcaaggaccttgaccatgactcatgcaagggtgtttatgacaacattagcaagaacgccctattagtgtactactgttggatgtcagatactatgtctaaggcatctacttttgtatctttcgacctggattatattggttga

SEQ ID NO: 12 - DNA, 892 bp, synthetic construct
  misc_feature 1..892: note = Synthetic polynucleotide-MP homolog 1
  source 1..892: mol_type = other DNA
aagttaacatttattttaacgacttggcttgagaagcctgacaattattattaatacactcttggacagttgtcctgactagctcattcaactgtcccattgacattgttatgttggattccgctctctgggcccccacgatcgaagcagactctcctgggtctaggacgctggtccccagcctattcagatgtctgtatggatggagttcgttctccacctctgagtccgcatctgagtggcccgttcctattgtactcctggaagcccatgattctccaggccttatctcgattgggcctctcagcccaaccctggacatggacgcgcatctgatgggcttcctttcccatttcccatagtcgacgtgggagaagtccacatccttatcggtgaactgtttggacaggatcttgactgtcggtgcccggaatgggatatcgaccgagtgtttcgccgtcgatagcttcagcttccctttgaacttggcgaaatgggtcctctgatgaacattcgtatcgcacaccctgtaatagagcttccatggaattgggtccttgagcgagaagaatgaagccgagaaatagtggagatctatgttacatctgatcggaaatgtccacgacgcttgtaatgattcgttgtcagtcatcctcttgtcgtggatctccacaattaccgacccggtggcattaatcggcacttgctgtctgtattctatgacgcaatggtctatcttcatgcagctacgactgagtctagctgtcaattgcgacgccgtcgaaggaaattgcagtattatctcagttaggtcatgggaaagctgatactcgtcccggtgagactctatgtagttaaaggcactcggaggatttactaactgagaatccat

SEQ ID NO: 13 - DNA, 881 bp, synthetic construct
  misc_feature 1..881: note = Synthetic polynucleotide-MP homolog 2
  source 1..881: mol_type = other DNA
tactgcaatgatttgggctgtgaaggtctacaattactattgatacactcctggacagtagtcctaacgagctcgttcaactggcccattgacatcgtgatgttggactccgctctctgggctcctataactgacgcagactctcctgggtctagaacggtggtcccgagcctgctcaggtgtctgtaagggtggagttcgttctccacctccgagtccgcctctgaatggcccgttcctatggtgctcctggaagcccacgattcaccaggccttatctctattgggcctcgaagcccaacccttgacatggacgcgcatctgatgggcttcctttcccatcttccgtagtcgacatgggaaaagtccacatctttgtccgtgaactgtttggacaagatctttactgttggtgcccggaaagggatgtcgacggagtgtttcgccgtcgataatttcagcttccctttgaacttcgcgaagtgggtcctctgatgaacattcgtgtcgcaaactctgtaatagagtttccatggaattgggtccttgagcgagaagaatgaagctgaaaagtagtggagatctatgttgcatctgatcggaaaagtccacgacgcctgcagtgactcgttgtccgtcatcctcttgtcgtggatctccacaattaccgaccccgtcgcgttgatcggaacttgctgcctgtattctatgacgcagtggtcgatcttcatgcagctacgactgagtctagccgtcaactgagacgccgtcgacggaaattgcagaacgatctcagttaggtcatgcgaaagctgatattcgtccctatgggactctatgtaattgaaggcatgcggaggattaaccaactgagaatccat

SEQ ID NO: 14 - protein, 7 aa, synthetic construct
  REGION 1..7: note = Synthetic peptide
  VAR_SEQ 7
ENLYFQX

SEQ ID NO: 15 - protein, 7 aa, synthetic construct
  REGION 1..7: note = Synthetic peptide
ENLYFQG